SEQUENCE LISTING (5 sequences)

SEQ ID NO: 1 - protein, 76 aa, Homo sapiens [sequence omitted]

SEQ ID NO: 2 - protein, 6 aa, Artificial
  synthesized tag sequence
HHHHHH

SEQ ID NO: 3 - protein, 1058 aa, Homo sapiens
MSSSPLSKKRRVSGPDPKPGSNCSPAQSVLSEVPSVPTNGMAKNGSEADIDEGLYSRQLYVLGHEAMKRLQTSSVLVSGLRGLGVEIAKNIILGGVKAVTLHDQGTAQWADLSSQFYLREEDIGKNRAEVSQPRLAELNSYVPVTAYTGPLVEDFLSGFQVVVLTNTPLEDQLRVGEFCHNRGIKLVVADTRGLFGQLFCDFGEEMILTDSNGEQPLSAMVSMVTKDNPGVVTCLDEARHGFESGDFVSFSEVQGMVELNGNQPMEIKVLGPYTFSICDTSNFSDYIRGGIVSQVKVPKKISFKSLVASLAEPDFVVTDFAKFSRPAQLHIGFQALHQFCAQHGRPPRPRNEEDAAELVALAQAVNARALPAVQQNNLDEDLIRKLAYVAAGDLAPINAFIGGLAAQEVMKACSGKFMPIMQWLYFDALECLPEDKEVLTEDKCLQRQNRYDGQVAVFGSDLQEKLGKQKYFLVGAGAIGCELLKNFAMIGLGCGEGGEIIVTDMDTIEKSNLNRQFLFRPWDVTKLKSDTAAAAVRQMNPHIRVTSHQNRVGPDTERIYDDDFFQNLDGVANALDNVDARMYMDRRCVYYRKPLLESGTLGTKGNVQVVIPFLTESYSSSQDPPEKSIPICTLKNFPNAIEHTLQWARDEFEGLFKQPAENVNQYLTDPKFVERTLRLAGTQPLEVLEAVQRSLVLQRPQTWADCVTWACHHWHTQYSNNIRQLLHNFPPDQLTSSGAPFWSGPKRCPHPLTFDVNNPLHLDYVMAAANLFAQTYGLTGSQDRAAVATFLQSVQVPEFTPKSGVKIHVSDQELQSANASVDDSRLEELKATLPSPDKLPGFKMYPIDFEKDDDSNFHMDFIVAASNLRAENYDIPSADRHKSKLIAGKIIPAIATTTAAVVGLVCLELYKVVQGHRQLDSYKNGFLNLALPFFGFSEPLAAPRHQYYNQEWTLWDRFEVQGLQPNGEEMTLKQFLDYFKTEHKLEITMLSQGVSMLYSFFMPAAKLKERLDQPMTEIVSRVSKRKLGRHVRALVLELCCNDESGEDVEVPYVRYTIR

SEQ ID NO: 4 - protein, 118 aa, Homo sapiens
MFHWQATIMGPNDSPYQGGVFFLTIHFPTDYPFKPPKVAFTTRIYHPNINSNGSICLDILRSQWSPALTISKVLLSICSLLCDPNPDDPLVPEIARIYKTDREKYNRIAREWTQKYAM

SEQ ID NO: 5 - protein, 643 aa, Homo sapiens
MGQESSKPVWPNPTGGYQSNTGRRYGRRHAYVSFRPPTSQRERIASQRKTNSEVPMHRSAPSQTTKRSRSPFSTTRRSWDDSESSGTNLNIDNEDYSRYPPREYRASGSRRGMAYGHIDSYGADDSEEEGAGPVERPPVRGKTGKFKDDKLYDPEKGARSLAGPPPHFSSFSRDVREERDKLDPVPAARCSASRADFLPQSSVASQSSSEGKLATKGDSSERERREQNLPARPSRAPVSICGGGENTSKSAEEPVVRPKIRNLASPNCVKPKIFFDTDDDDDMPHSTSRWRDTANDNEGHSDGLARRGRGESSSGYPEPKYPEDKREARSDQVKPEKVPRRRRTMADPDFWTHSDDYYKYCDEDSDSDKEWIAALRRKYRSREQTLSSSGESWETLPGKEEREPPQAKVSASTGTSPGPGASASAGAGAGASAGSNGSNYLEEVREPSLQEEQASLEEGEIPWLQYHENDSSSEGDNDSGHELMQPGVFMLDGNNNLEDDSSVSEDLEVDWSLFDGFADGLGVAEAISYVDPQFLTYMALEERLAQAMETALAHLESLAVDVEVANPPASKESIDALPEILVTEDHGAVGQEMCCPICCSEYVKGEVATELPCHHYFHKPCVSIWLQKSGTCPVCRCMFPPPL

(19) United States
(12) Patent Application Publication
(10) Pub. No.: US 2006/0088901 A1
(43) Pub. Date: Apr. 27, 2006

(54) Methods and kits for determining ubiquitin protein ligase (E3) activity

(76) Inventors: Zhuangwu Li, Bethesda, MD (US); Hue Mao, Hacienda Hgts., CA (US)

Correspondence Address: Zhuangwu Li, Suite #19, 10075 Tyler Place, Ijamsville, MD, 21754, US

(21) Appl. No.: 11/251,673

(22) Filed: Oct. 17, 2005

Related U.S. Application Data
(60) Provisional application No. 60/621,009, filed on Oct. 21, 2004.

(51) Int. Cl. C12Q 1/37

(52) U.S. Cl. 435/23

(57) ABSTRACT

The present invention provides a method and an assay kit for detecting ubiquitin ligase (E3) activity in a sample. The method includes the steps of (1) preparing a sample mixture comprising ubiquitin, a ubiquitin activating enzyme (E1), a ubiquitin conjugating enzyme (E2), a reaction buffer, and the sample; (2) incubating the sample mixture under conditions suitable for ubiquitination to occur; and (3) detecting a presence of ubiquitination products in the sample mixture after the incubation.

### RELATED APPLICATION

This application claims priority to Application Ser. No. 60/621,009, filed Oct. 21, 2004, entitled “Method for determining ubiquitin protein ligase activity.”

### TECHNICAL FIELD

The present invention generally relates to biotechnology. In particular, the present invention relates to a method for determining ubiquitin protein ligase activity.

### BACKGROUND

Many cellular functions are directly or indirectly regulated by covalent attachment of ubiquitin to a target protein, a process called ubiquitination. Ubiquitin is a highly conserved 76 amino acid protein expressed in all eukaryotic cells. The attachment of ubiquitin to a target protein results in a poly-ubiquitinated target protein that is rapidly detected and degraded by the 26S proteasome.

The ubiquitination process is catalyzed by three enzymes, namely, ubiquitin activating enzyme (E1), ubiquitin conjugating enzyme (E2), and ubiquitin protein ligase (E3). Ubiquitin is first activated in an adenosine triphosphate (ATP)-dependent manner by E1. The C-terminus of a ubiquitin forms a high energy thiolester bond with E1. The ubiquitin is then passed to E2 and is linked to this enzyme via a thiolester bond. The ubiquitin is finally linked to its target protein to form a terminal isopeptide bond under the guidance of E3. In other words, E3 determines the substrate-specificity of ubiquitination process. In this process, chains of ubiquitin are formed on the target protein, each covalently ligated to the next through the activity of E3, which largely determines to which protein ubiquitin is attached.

Ubiquitination regulates cellular functions by targeting protein for degradation by the proteasomes. It also regulates cell cycle, kinase activity, transcription factor activation, DNA repair, and protein trafficking. Aberrant ubiquitination is involved in the pathogenesis of many diseases, such as cancer, cardiovascular diseases, and neurodegenerative diseases. In fact, ubiquitin-proteasome pathway has become a very attractive drug target for the treatment of many diseases. In May of 2003, Velcade, the first anti-cancer drug directed against proteasome activity was approved by FDA. Being more specific, ubiquitin ligases are bound to be more attractive targets for drug development. Although ubiquitination requires three enzymes (E1, E2, E3), the whole process can be biochemically reconstituted in vitro. There are one E1, about 60 E2s, and hundreds of E3s in human genome database. However, fewer than 10% of E3s have been studied or even tested for E3 activity. There is no kit currently available on the market that allows easy detection of E3 activity. The present invention is developed to satisfy this need in scientific community.

### SUMMARY

One aspect of the present invention relates to a method for detecting E3 activity in a sample. The method comprises the steps of preparing a sample mixture comprising ubiquitin, a ubiquitin activating enzyme (E1), a ubiquitin conjugating enzyme (E2), a reaction buffer, and the sample; preparing a control mixture comprising said E1, said E2, a ubiquitin ligase (E3), and said buffer; incubating said sample mixture and said control mixture under conditions suitable for ubiquitination to occur; and detecting a presence of ubiquitination product in said sample mixture and in said control mixture after the incubation. The method may further contain the steps of quantifying the ubiquitination product in the sample mixture and control mixture after incubation; and determining E3 activity in the sample.

In an embodiment, the ubiquitin activating enzyme (E1) is a bacteria lysate having E1 activity. In another embodiment, the ubiquitin conjugating enzyme (E2) is a bacteria lysate having UbCH5b activity. In another embodiment, the ubiquitin is labeled with a histidine (His)-tag. In yet another embodiment, the sample mixture further comprises a ubiquitination substrate protein.

Another aspect of the present invention relates to a kit for detecting ubiquitin protein ligase (E3) activity in a sample. The kit comprises a ubiquitin activating enzyme (E1), a ubiquitin conjugating enzyme (E2), a ubiquitin, a control ubiquitin protein ligase (E3), a reaction buffer and an instruction for assay procedure.

In an embodiment, the ubiquitin conjugating enzyme (E2) in the kit is a bacteria lysate having UbCH5b activity. In another embodiment, the ubiquitin in the kit is labeled with a His-tag. In yet another embodiment, the kit further comprises at least one detection agent selected from the group consisting of anti-ubiquitin antibodies, anti-Flag antibodies, anti-myc antibodies, anti-T7 antibodies, anti-HA antibodies, nickel-HRP, and enzymatic chemiluminescence (ECL) substrates.

### BRIEF DESCRIPTION OF THE FIGURES

FIG. 1 is the picture of an Sodium Dodecyl (lauryl) Sulfate-Polyacrylamide Gel Electrophoresis (SDS-PAGE) showing the detection of E3 activity

### DESCRIPTION OF THE INVENTION

The present invention relates to a method for determining E3 activity. The method can be used, for example, to rapidly determine E3 activity from patient samples for the diagnoses of E3-related diseases. The method contains the steps of preparing a sample mixture comprising ubiquitin, E1, E2, a reaction buffer, and the sample; preparing a control mixture comprising said E1, said E2, a ubiquitin ligase (E3), and the reaction buffer; incubating the sample mixture and control mixture under conditions suitable for ubiquitination to occur; and detecting the presence of ubiquitination product in the sample mixture and control mixture after the incubation. The method may further contain the steps of quantifying the ubiquitination product in the sample mixture and control mixture after incubation, and determining E3 activity in the sample.

Ubiquitin

In one embodiment, the present invention utilizes a 76 amino acid human ubiquitin having the amino acid sequence of that depicted in American Type Culture Collection (ATCC) accession number P02248 (SEQ ID NO:1), which is incorporated herein by reference. ATCC accession numbers are found in Genbank. Sequences of GenBank accession numbers are incorporated herein by reference. GenBank is known in the art, see, e.g., Benson, DA, et al., Nucleic Acids Research 26:1-7 (1998) and http://www.ncbi.nlm.nih.gov/.

Also encompassed by “ubiquitin” is naturally occurring alleles and man-made variants of such a 76 amino acid polypeptide. In an embodiment, variants of ubiquitin have an overall amino acid sequence identity of greater than about 75%, preferably greater than about 80%, more preferably greater than about 85% and even more preferably greater than 90% of the amino acid sequence recited in SEQ ID NO:1. In some embodiments the sequence identity will be as high as about 93 to 95 or 98%.

As is known in the art, a number of different programs can be used to identify whether a protein (or nucleic acid as discussed below) has sequence identity or similarity to a known sequence. Sequence identity and/or similarity is determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.; the BLAST algorithm, described in Altschul et al., J. Mol. Biol. 215, 403-410, (1990) and Karlin et al., PNAS USA 90:5873-5787 (1993). and gapped BLAST as reported by Altschul et al. Nucleic Acids Res. 25:3389-3402s.

Ubiquitin proteins of the present invention may be shorter than the amino acid sequence recited in SEQ ID NO:1. Thus, in a preferred embodiment, included within the definition of ubiquitin are portions or fragments of the amino acid sequence recited in SEQ ID NO:1. Fragments of ubiquitin are considered ubiquitin proteins if they are ligated to another polypeptide by ubiquitin ligase enzymes.

Ubiquitin proteins of the present invention may also be made longer than the amino acid sequence recited in SEQ ID NO:1; for example, by the addition of tags, the addition of other fusion sequences, or the elucidation of additional coding and non-coding sequences. As described below, the fusion of a ubiquitin peptide to a 6-His tag (SEQ ID NO:2) is particularly preferred.

By “tag” is meant an attached molecule or molecules useful for the identification or isolation of the attached component. For example, a ubiquitin comprising a tag is referred to herein as “tagged ubiquitin”. Preferably, the tag is covalently bound to the attached component. Components may comprise more than one tag. Preferred tags include, but are not limited to, histidine (His), myc, T7, HA, and Flag. As will be evident to the skilled artisan, many molecules may find use as more than one type of tag, depending upon how the tag is used. A tag may directly or indirectly binds to a label.

By “label” is meant a molecule that can be directly (i.e., a primary label) or indirectly (i.e., a secondary label) detected; for example a label can be visualized and/or measured or otherwise identified so that its presence or absence can be known. As will be appreciated by those in the art, the manner in which this is done will depend on the label. Preferred labels include, but are not limited to, label enzymes, fluorescent labels, and radioisotope labels.

The label enzyme can be any enzyme that may be reacted in the presence of a label enzyme substrate and produces a detectable product. Suitable label enzymes for use in the present invention include but are not limited to, horseradish peroxidase (HRP), alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

The fluorescent label can be any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green. Suitable optical dyes are described in the 1996 Molecular Probes Handbook by Richard P. Haugland, hereby expressly incorporated by reference. Suitable fluorescent labels also include, but are not limited to, green fluorescent protein (GFP; Chalfie, et al., Science 263(5148):802-805 (Feb. 11, 1994); and EGFP; Clontech-Genbank Accession Number U55762), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), luciferase (Ichiki, et al., J. Immunol. 150(12):5408-5417 (1993)), .beta.-galactosidase (Nolan, et al., Proc Natl Acad Sci USA 85(8):2603-2607 (April 1988)) and Renilla WO 92/15673; WO 95/07463; WO 98/14605; WO 98/26277; WO 99/49019; U.S. Pat. Nos. 5,292,658; 5,418,155; 5,683,888; 5,741,668; 5,777,079; 5,804,387; 5,874,304; 5,876,995; and 5,925,558) All of the above-cited references are expressly incorporated herein by reference.

The radioisotope label can be any radioactive molecule. Suitable radioisotopes for use in the invention include, but are not limited to 14C, 3H, 32P, 33P, 35S, 125I, and 131I. The use of radioisotopes as labels is well known in the art.

It is important to remember that ubiquitin is ligated to substrate protein by its terminal carboxyl group to a lysine residue, including lysine residues on other ubiquitin. Therefore, attachment of tags or labels should not interfere with either of these active groups on the ubiquitin. Amino acids may be added to the sequence of protein, through means well known in the art and described herein, for the express purpose of providing a point of attachment for a label.

Typically, the tag forms a binding pair with a labeled or unlabeled binding partner. Suitable binding pairs for use in the invention include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine/anti-rhodamine), poly-histidine/nickel, biotin/avidin (or biotin/streptavidin) and calmodulin binding protein (CBP)/calmodulin. Other suitable binding pairs include polypeptides such as the FLAG-peptide (DYKDDDDK; SEQ ID NO:16) [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA 87:6393-6397 (1990)] and the antibodies each thereto. Generally, in a preferred embodiment, the smaller of the binding pair partners serves as the tag, as steric considerations in ubiquitin ligation may be important. As will be appreciated by those in the art, binding pair partners may be used in applications other than for labeling, as is further described below.

As will be appreciated by those in the art, a partner of one binding pair may also be a partner of another binding pair. For example, an antigen (first moiety) may bind to a first antibody (second moiety) which may, in turn, be an antigen for a second antibody (third moiety). It will be farther appreciated that such a circumstance allows indirect binding of a first moiety and a third moiety via an intermediary second moiety that is a binding pair partner to each.

As will be appreciated by those in the art, a partner of a binding pair may comprise a label, as described above. It will further be appreciated that this allows for a tag to be indirectly labeled upon the binding of a binding partner comprising a label. For example, a His tag may be indirectly labeled with nickel-HRP.

As will be appreciated by those in the art, tag-components of the invention can be made in various ways, depending largely upon the form of the tag. Components of the invention and tags are preferably attached by a covalent bond.

The production of tag-polypeptides by recombinant means when the tag is also a polypeptide is well known in the art. For example, production of proteins having His-tags by recombinant means is well known, and kits for producing such proteins are commercially available. Such a kit and its use is described in the QIA express Handbook from Quiagen by Joanne Crowe et al., hereby expressly incorporated by reference. Production of Flag-labeled proteins is also well known in the art and kits for such production are commercially available from Kodak and Sigma. Methods for the production and use of Flag-labeled proteins are found, for example, in Winston et al., Genes and Devel. 13:270-283 (1999), incorporated herein in its entirety, as well as product handbooks provided with the above-mentioned kits.

Biotinylation of target molecules and substrates is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known (supra).

Methods for labeling of proteins with radioisotopes are known in the art. For example, such methods are found in Ohta et al., Molec. Cell 3:535-541 (1999), which is hereby incorporated by reference in its entirety.

The functionalization of labels with chemically reactive groups such as thiols, amines, carboxyls, etc. is generally known in the art. In a preferred embodiment, the tag is functionalized to facilitate covalent attachment.

The covalent attachment of the tag may be either direct or via a linker. In one embodiment, the linker is a relatively short coupling moiety, that is used to attach the molecules. A coupling moiety may be synthesized directly onto a component of the invention, ubiquitin for example, and contains at least one functional group to facilitate attachment of the tag. Alternatively, the coupling moiety may have at least two functional groups, which are used to attach a functionalized component to a functionalized tag, for example. In an additional embodiment, the linker is a polymer. In this embodiment, covalent attachment is accomplished either directly, or through the use of coupling moieties from the component or tag to the polymer. In a preferred embodiment, the covalent attachment is direct, that is, no linker is used. In this embodiment, the component preferably contains a functional group such as a carboxylic acid which is used for direct attachment to the functionalized tag. It should be understood that the component and tag may be attached in a variety of ways, including those listed above. What is important is that manner of attachment does not significantly alter the functionality of the component. For example, in tag-ubiquitin, the tag should be attached in such a manner as to allow the ubiquitin to be covalently bound to other ubiquitin to form polyubiquitin chains. As will be appreciated by those in the art, the above description of covalent attachment of a label and ubiquitin applies equally to the attachment of virtually any two molecules of the present disclosure.

In a preferred embodiment, ubiquitin is in the form of tag-ubiquitin, wherein, tag is a partner of a binding pair. Preferably in this embodiment the tag is a His tag (SEQ ID NO:2) and the binding-partner is nickel-HRP.

Ubiquitin Activating Enzyme (E1)

E1 proteins useful in the invention include, but are not limited to, mammalian E1 (e.g., human, mouse and rat E1, etc.) or Els of other species such as plant (e.g., wheat), zebrafish and C. elegans. In a preferred embodiment, E1 is a human E1 having the amino acid sequence recited in SEQ ID NO:3. E1 is commercially available from Affiniti Research Products (Exeter, U.K.). Variants of the cited E1 proteins, also included in the term “E1”, can be made as described herein.

Ubiquitin Conjugating Enzyme (E2)

E2 proteins useful in the invention include, but are not limited to, mammalian E2 (e.g., human, mouse and rat E2s, etc.) or E2s of other species such as plant (e.g., wheat), zebrafish and C. elegans. The skilled artisan will appreciate that many different E2 proteins and isozymes are known in the filed and may be used in the present invention, provided that the E2 has ubiquitin conjugating activity. Also specifically included within the term “E2” are variants of E2, which can be made as described herein. In a preferred embodiment, the E2 protein is UbCH5b with the amino acid sequence shown in SEQ ID NO:4. While some E2s are E3-specific, i.e., they work with only certain E3s, UbCH5b works with most of the known E3s. In another embodiment, the E2 of the present invention contains a panel of E2 proteins. In a preferred embodiment, the panel of E2s contains 2-20 E2 proteins. In a more preferred embodiment, the panel of E2s contains 3-10 E2 proteins. The inclusion of multiple E2s in the assay system would allow the detection of most, if not all, E3 activities.

Ubiquitin Ligase (E3)

In the present invention, a known E3 or a panel of known E3s are utilized as controls to determine E3 activities in test samples. By “E3” is meant a ubiquitin ligase comprising one or more components associated with ligation of ubiquitin to a ubiquitination substrate protein for ubiquitin-dependent proteolysis. Suitable E3s include, but are not limited to, RING finger-, Homologous to the E6-AP Carboxyl Terminus (HECT)-, U box-, PHD finger-type of E3s. A preferred E3 is the human E3 having an amino acid sequence recited in SEQ ID NO:5. These E3 proteins and variants may be made as described herein. In a preferred embodiment, the E3 is a bacteria lysate having E3 activity.

Recombinant Proteins

The ubiquitin protein, E1, E2, and control E3 are preferably recombinant proteins. A “recombinant protein” is a protein made using recombinant techniques, i.e. through the expression of a recombinant nucleic acid as described below. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions.

As used herein and further defined below, “nucleic acid” may refer to either DNA or RNA, or molecules which contain both deoxy- and ribonucleotides. The nucleic acids include genomic DNA, cDNA and oligonucleotides including sense and anti-sense nucleic acids. Such nucleic acids may also contain modifications in the ribose-phosphate backbone to increase stability and half-life of such molecules in physiological environments.

The nucleic acid may be double stranded, single stranded, or contain portions of both double stranded or single stranded sequence. As will be appreciated by those in the art, the depiction of a single strand (“Watson”) also defines the sequence of the other strand (“Crick”); thus the sequences depicted in figures also include the complement of the sequence. By the term “recombinant nucleic acid” herein is meant nucleic acid, originally formed in vitro, in general, by the manipulation of nucleic acid by endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

The terms “polypeptide” and “protein” may be used interchangeably throughout this application and mean at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradation.

The Protein Variants

In one embodiment, the present invention provides compositions containing protein variants, for example ubiquitin, E1, and or E2 variants. These variants fall into one or more of three classes: substitutional, insertional or deletional variants. These variants ordinarily are prepared by site specific mutagenesis of nucleotides in the DNA encoding a protein of the present compositions, using cassette or polymerase chain reaction (PCR) mutagenesis or other techniques well known in the art, to produce DNA encoding the variant, and thereafter expressing the DNA in recombinant cell culture as outlined above. However, variant protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed variants screened for the optimal desired activity. Techniques for making substitution mutations at predetermined sites in DNA having a known sequence are well known, for example, M13 primer mutagenesis and PCR mutagenesis. Rapid production of many variants may be done using techniques such as the method of gene shuffling, whereby fragments of similar variants of a nucleotide sequence are allowed to recombine to produce new variant combinations. Examples of such techniques are found in U.S. Pat. Nos. 5,605,703; 5,811,238; 5,873,458; 5,830,696; 5,939,250; 5,763,239; 5,965,408; and 5,945,325, each of which is incorporated by reference herein in its entirety. Screening of the mutants is done using ubiquitin ligase activity assays of the present invention.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the protein are desired, substitutions are generally made in accordance with Table 1:

TABLE I

| Original Residue | Exemplary Substitutions |
|---|---|
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the proteins as needed. Alternatively, the variant may be designed such that the biological activity of the protein is altered. For example, glycosylation sites may be altered or removed.

Covalent modifications of polypeptides are included within the scope of this invention. One type of covalent modification includes reacting targeted amino acid residues of a polypeptide with an organic derivatizing agent that is capable of reacting with selected side chains or the N- or C-terminal residues of a polypeptide. Derivatization with bifunctional agents is useful, for instance, for crosslinking a protein to a water-insoluble support matrix or surface for use in the method for screening assays, as is more fully described below. Commonly used crosslinking agents include, e.g., 1,1-bis(diazoacetyl)-2-phenylethane, glutaraldehyde, -hydroxy-succinimide esters, for example, esters with 4-azidosalicylic acid, homobifunctional imidoesters, including disuccinimidyl esters such as 3,3′-dithiobis(succinimidyl-propionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the alpha.-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of a polypeptide included within the scope of this invention comprises altering the native glycosylation pattern of the polypeptide. “Altering the native glycosylation pattern” is intended for purposes herein to mean deleting one or more carbohydrate moieties found in native sequence polypeptide, and/or adding one or more glycosylation sites that are not present in the native sequence polypeptide.

Addition of glycosylation sites to polypeptides may be accomplished by altering the amino acid sequence thereof. The alteration may be made, for example, by the addition of, or substitution by, one or more serine or threonine residues to the native sequence polypeptide (for O-linked glycosylation sites). The amino acid sequence may optionally be altered through changes at the DNA level, particularly by mutating the DNA encoding the polypeptide at preselected bases such that codons are generated that will translate into the desired amino acids.

Another means of increasing the number of carbohydrate moieties on a polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of a protein comprises linking the polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Proteins/polypeptides of the present invention may also be modified in a way to form chimeric molecules comprising a first polypeptide fused to another, heterologous polypeptide or amino acid sequence. In one embodiment, such a chimeric molecule comprises a fusion of a ubiquitin polypeptide (or an E2 or an E3, as defined below) with a tag polypeptide which provides an epitope to which an anti-tag antibody can selectively bind. The epitope tag is generally placed at the amino- or carboxyl-terminus of the polypeptide. The presence of such epitope-tagged forms of a polypeptide can be detected using an agent that binds specifically to the tag polypeptide. Also, providing an epitope tag enables the polypeptide to be readily purified by affinity purification using an anti-tag antibody or another type of affinity matrix that binds to the epitope tag. In an alternative embodiment, the chimeric molecule may comprise a fusion of a polypeptide disclosed herein with an immunoglobulin or a particular region of an immunoglobulin. For a bivalent form of the chimeric molecule, such a fusion could be to the Fc region of an IgG molecule. Tags for components of the invention are defined and described in detail below.

Proteins/polypeptides of the present invention may be produced by culturing a host cell transformed with an expression vector containing nucleic acid encoding the protein, under the appropriate conditions to induce or cause expression of the protein. The conditions appropriate for protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect and animal cells, including mammalian cells. Of particular interest are Drosophila melanogaster cells, Pichia pastoris and P. methanolica, Saccharomyces cerevisiae and other yeasts, E. coli, Bacillus subtilis, SF9 cells, SF21 cells, C129 cells, Saos-2 cells, Hi-5 cells, 293 cells, Neurospora, BHK, CHO, COS, and HeLa cells. Of greatest interest are Pichia pastoris and P. methanolica, E. coli, SF9 cells, SF21 cells and Hi-5 cells.

In a preferred embodiment, the proteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for a protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, proteins are expressed in bacterial systems. Bacterial expression systems are well known in the art.

A suitable bacterial promoter is any nucleic acid sequence capable of binding bacterial RNA polymerase and initiating the downstream (3′) transcription of the coding sequence of a protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan.

Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgamo (SD) sequence and includes an initiation codon and a sequence 3-9 nucleotides in length located 3-11 nucleotides upstream of the initiation codon.

The expression vector may also include a signal peptide sequence that provides for secretion of the protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

These components are assembled into expression vectors. Expression vectors for bacteria are well known in the art, and include vectors for Bacillus subtilis, E. coli, Streptococcus cremoris, and Streptococcus lividans, among others.

The bacterial expression vectors are transformed into bacterial host cells using techniques well known in the art, such as calcium chloride treatment, electroporation, and others.

In one embodiment, proteins are produced in insect cells. Expression vectors for the transformation of insect cells, and in particular, baculovirus-based expression vectors, are well known in the art.

In another embodiment, proteins are produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii P. methanolica and P. pastoris, Schizosaccharomyces pombe, and Yarrowia lipolytica. Preferred promoter sequences for expression in yeast include the inducible GAL 1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

The protein may also be made as a fusion protein, using techniques well known in the art. Thus, for example, the protein may be made as a fusion protein to increase expression, or for other reasons. For example, when the protein is a peptide, the nucleic acid encoding the peptide may be linked to other nucleic acid for expression purposes. Similarly, proteins of the invention can be linked to protein labels, such as green fluorescent protein (GFP), red fluorescent protein (RFP), blue fluorescent protein (BFP), yellow fluorescent protein (YFP), etc.

In an embodiment, the protein is purified or isolated after expression. Proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase high performance liquid chromatography (HPLC), and chromatofocusing. For example, the ubiquitin protein may be purified using a standard anti-ubiquitin antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the protein. In some instances no purification will be necessary.

Samples

The sample to be tested for E3 activity can be any biological sample such as body fluid samples, tissue samples and cell samples. In one embodiment, tissue or cell sample is lysed in a lysis buffer prior to the addition of E1, E2 and the reaction buffer. In another embodiment, the sample is directly mixed with E1, E2 and the reaction buffer without prior lysis. The samples can also be purified or unpurified proteins or polypepetides.

Ubiquitination Substrate Protein

Embodiments of the present invention involve binding ubiquitin to a ubiquitination substrate protein. By “ubiquitination substrate protein” is meant a protein to which the E3 can catalyze the covalent binding of ubiquitin and includes target proteins and ubiquitin. In a preferred embodiment, the ubiquitination substrate protein is ubiquitin itself and the ubiquitin ligase catalyzes the formation of polyubiquitin chains. In other words, the polyubiquitin chains are formed by the ubiquitin ligase in the absence of any target protein.

Reaction Conditions

The components of the invention are combined under reaction conditions that favor ubiquitin ligase activity and/or ubiquitination acitivty. Conditions for ubiquitination are well-known in the art (Jensen J P et al. JBC 270: 30408(1995)); Chen A et al. JBC, 277: 22085(2002). Typically, the ubiquitination is carried out in a buffer with a pH range of 7.0-8.0, preferably from pH7.2-7.6. Suitable buffer systems include, but are not limited to, phosphate buffered saline (PBS), tris(hydroxymethyl)methylamine (TRIS) and 4-2-hydroxyethyl-1-piperazineethanesulfonic acid (HEPES). In a preferred embodiment, the buffer contains 1-20 mM of Mg²⁺ and 0.5-10 mM of ATP. In a more preferred embodiment, the buffer contains 40 mM TRIS, pH 7.6, 5 mM MgCl2, 2 mM ATP, and 2 mM DTT (dithiothreitol). Incubations may be performed at any temperature which facilitates optimal activity, typically between 4° C. and 40° C., preferably at room temperature or 30° C. Incubation periods are selected for optimum activity, but may also be optimized to facilitate rapid high through put screening. Typically, an incubation time of between 0.5 and 2.5 hours is sufficient.

The components (i.e., ubiquitin, E1, E2, the sample, ect.) may be mixed in any order that promotes ubiquitin ligase activity. In a preferred embodiment, ubiquitin is provided in a reaction buffer solution, followed by addition of E1, E2 and the sample.

Detection of Ubiquitination Product

The ubiquitination products (ubiquitinized substrate or polyubiquitin) can be detected using techniques well-known in the art. As will be understood by one of ordinary skill in the art, the mode of measuring will depend on the specific tag attached to the ubiquitin. If the ubiquitin is not tagged, the ubiquitinized substrate or polyubiquitin can be detected using an anti-ubiquitin antibody followed by standard immuno-detection techniques.

Commonly used detection techniques include enzyme-mediated, fluorescence/luminescence-mediated, and radioisotope-mediated detection. As used herein, “luminescence” or “fluorescence” means photon emission from a fluorescent label. Equipment for such measurement is commercially available and easily used by one of ordinary skill in the art to make such a measurement. Radioisotope labeling may be measured by scintillation counting, or by densitometry after exposure to a photographic emulsion, or by using a device such as a Phosphorlmager or densitometer.

It is understood by the skilled artisan that it may be necessary to separate the ubiquitination products from free ubiquitin. The separation can be achieved using standard procedures such as SDS-PAGE and HPLC.

Determining E3 Activity in the Sample

The E3 activity in the sample can be quantified using standard plotting methods well known to one skilled in the art. Briefly, a set of control mixtures are prepared using various amounts of control E3 to produce a standard curve, which is then used for the determination of E3 activity in the sample mixture.

The skilled artisan would understand that the steps of the assays provided herein can vary in order. It is also understood, however, that while various options (of compounds, properties selected or order of steps) are provided herein, the options are also each provided individually, and can each be individually segregated from the other options provided herein. Moreover, steps that are obvious and known in the art that will increase the sensitivity of the assay are intended to be within the scope of this invention. For example, there may be additionally washing steps, blocking steps, etc.

Cloning and Expression of New E3 Proteins

The present invention provides methods for determining ubiquitin ligase (E3) activity in a sample. When E3 activity is detected in the sample, the enzyme may be cloned and expressed using standard molecular cloning and expression techniques. Probe or degenerate polymerase chain reaction (PCR) primer sequences may be used to find other related ubiquitination proteins from humans or other organisms. As will be appreciated by those in the art, particularly useful probe and/or PCR primer sequences include the unique areas of a nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art. It is therefore also understood that provided along with the sequences in the sequences cited herein are portions of those sequences, wherein unique portions of 15 nucleotides or more are particularly preferred. The skilled artisan can routinely synthesize or cut a nucleotide sequence to the desired length.

Once isolated from its natural source, e.g., contained within a plasmid or other vector or excised therefrom as a linear nucleic acid segment, the recombinant nucleic acid can be further-used as a probe to identify and isolate other nucleic acids. It can also be used as a “precursor” nucleic acid to make modified or variant nucleic acids and proteins.

Using the nucleic acids of the present invention which encode a protein, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. As another example, operably linked refers to DNA sequences linked so as to be contiguous, and, in the case of a secretory leader, contiguous and in reading fram. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the protein; for example, transcriptional and translational regulatory nucleic acid sequences from Bacillus are preferably used to express the protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

Detection Kit

Another aspect of the present invention provides a kit for detecting E3 activity in a sample. In an embodiment, the kit comprises a ubiquitin, a ubiquitin activating enzyme (E1), a ubiquitin conjugating enzyme (E2), a control ubiquitin ligase (E3), a reaction buffer, and an instruction for assay procedure. The kit may be used for diagnosing ubiquitin ligase-related diseases.

The ubiquitin may be tagged or untagged. In a preferred embodiment, the ubiquitin is tagged. In a more preferred embodiment, the ubiquitin is tagged with a His tag. In another embodiment, the E1 and E2 are bacteria extract. In a preferred embodiment, the E2 is a bacteria extract having UbCH5b activity.

The reaction buffer may include reagents like salts, solvents, buffers, neutral proteins, e.g. albumin, detergents, etc. The reaction buffer facilitates optimal ubiquitination enzyme activity and/or reduce non-specific or background interactions. The reaction buffer may also include reagents that otherwise improve the efficiency of the assay, such as protease inhibitors, nuclease inhibitors, anti-microbial agents, etc. In a preferred embodiment, the reaction buffer includes adenosine tri-phosphate (ATP).

In an embodiment, the assay kit further comprises a ubiquitination substrate protein. In another embodiment, the kit further comprises at least one detection agent selected from the group consisting of anti-ubiquitin antibodies, anti-Flag antibodies, anti-myc antibodies, anti-T7 antibodies, anti-HA antibodies, nickel-HRP, and ECL substrates. In yet another embodiment, the kit further comprises a sample lysis buffer. The lysis buffer may contain a suitable detergent, include but not limited to, Triton X-100 and NP40.

The components of the assay kit may be combined in varying amounts. In a preferred embodiment, ubiquitin is combined at a final concentration of from 20 to 200 ng per 100 ul reaction solution, most preferable at about 100 ng per 100 ul reaction solution.

In a preferred embodiment, the composition comprises E1 at a final concentration of from 1 to 50 ng per 100 ul reaction solution, more preferably from 1 ng to 20 ng per 100 ul reaction solution, most preferably from 5 ng to 10 ng per 100 ul reaction solution.

In a preferred embodiment, the composition comprises E2 at a final concentration of 10 to 100 ng per 100 ul reaction solution, more preferably 10-50 ng per 100 ul reaction solution.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are expressly incorporated by reference in their entirety.

### EXAMPLES

### Example 1

### E3 Activity Detection Kit

An assay kit is produced for detection of ubiquitin ligase (E3) activity in vitro. The kit contains the following component and can be used to detect self-ubiquitination and may be used to detect substrate ubiquitination.

Kit content (30 reactions):

| | |
|---|---|
| E1 lysate | 60 ul |
| E2 lysate | 60 ul |
| His-tagged ubiquitin lysate | 60 ul |
| Positive control E3 lysate | 60 ul |
| 10 X Reaction buffer | 30 ul |
| Nickel-HRP | 10 ul |

The E1 lysate is a bacteria lysate containing human E1 (SEQ ID NO:3) activity. The E2 lysate is a bacteria lysate containing human UbCH5b (SEQ ID NO:4) activity, which is known to support most E3 activities in vitro, including all known classes of E3s, such as RING finger, U box, and HECT domain E3s, originated from virus, yeast, plant, and mammals. Therefore, the kit is suitable for activity detection of any potential E3. The ubiquitin is human ubiquitin (SEQ ID NO:1) tagged with a his-tag (SEQ ID NO:2). The positive control E3 lysate is a bacterial lysate containing a human E3 (SEQ ID NO;5) activity. The 10× reaction buffer contains 400 mM Tris, pH 7.6, 50 mM MgCl₂, 20 mM ATP, and 20 mM DTT.

Components required but not included in the kit are SDS-PAGE apparatus, SDS-PAGE supplies, and enhanced chemilumilescence (ECL) substrate.

### Example 2

### E3 Assay Procedure

(A) Self-ubliquitination

1. Positive control E3 reaction:

| | |
|---|---|
| E1 lysate | 2 ul |
| E2 lysate | 2 ul |
| His-tagged ubiquitin lysate | 2 ul |
| Positive control E3 lysate | 3 ul |
| 10 X Reaction buffer | 2 ul |
| ddH2O | 9 ul |
| Gently mix and put on ice | |

2. Testing E3 reaction:

| | |
|---|---|
| E1 lysate | 2 ul |
| E2 lysate | 2 ul |
| His-tagged ubiquitin lysate | 2 ul |
| Testing E3 lysate or IPed E3 | 3 ul |
| 10 X Reaction buffer | 2 ul |
| ddH2O | 9 ul |

Note:
also prepare reactions with omission of E3, E1, E2, or ubiquitin as negative controls, assemble the reactions, gently mix and put on ice.

3. Incubate the reaction mix at 30° C. for up to 2 hours. The temperature can vary from RT to 37° C. and the incubation time can vary from 5 minutes to 2 hours depending on the E3 activity. Positive control E3 will show strong activity after 20 minutes incubation at 30° C. For initial testing, let the reaction proceed at 30°° C. for 2 hours is strongly recommended.
4. Stop the reaction by addition of SDS loading buffer and boil for 5 minutes.
5. SDS-PAGE and transfer to PVDF or Nitrocellulose membrane.
6. Blocking the membrane with 1% BSA in 1×PBS containing 0.1% Tween 20 (PBST) for 1 hour at room temperature.
7. Blotting the membrane with Ni-HRP (starting from 1:5000) in PBST for 1 hour at room temperature.
8. Washing with PBST 5 minutes×6.
9. Detection by ECL.
10. The E3 activity is reflected by laddering and/or smearing on the blot.

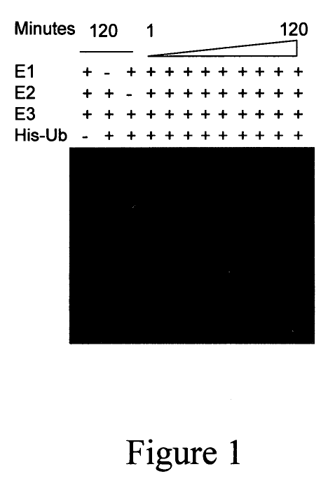

FIG. 1 is a picture of a Western blot showing detection of polyubiquitin following the assay procedure described above. From left to right, lanes 1-3 are negative controls, lanes 4-12 correspond to an incubation time of 0, 5, 10, 20, 30, 45, 60, and 120 minutes. As shown in FIG. 1, polyubiquitin is detectable after 5 minutes of incubation. One skilled in the art would understand that the membrane can be blotted with anti-ubiquitin or anti-His antibody following regular Western blotting procedure.

(B) Substrate Ubiquitination

Although this kit is not designed for substrate ubiquitination assay, it may be used in this aspect with some modifications of the protocol

1. Ubiquitination reaction:

| | | |
|---|---|---|
| E1 lysate | 2 | ul |
| E2 lysate | 2 | ul |
| His-tagged ubiquitin lysate | 2 | ul |
| Testing E3 lysate or IPed E3 | 3 | ul |
| Recombinant or IPed substrate | x | ul |
| 10 X Reaction buffer | 2 | ul |
| ddH2O up to | 20 | ul |

Note:
Also prepare reactions with omission of E3, E1, E2, or ubiquitin as negative controls, assemble the reactions, gently mix and put on ice. Increase the amounts of E1, E2, and E3 may increase the possibility for substrate ubiquitination

2. Incubate the reaction mix at 30° C. for 2 hours. * For initial testing, let the reaction proceed at 30° C. for 2 hours is strongly recommended.
3. Stop the reaction by addition of 8 ul 4×SDS loading buffer and boil for 5 minutes.
4. Adding 300 ul Triton X-100 lysis buffer and brief mix.
5. Immunoprecipitation with 1-5 ug anti-body against the substrate and 30 ul protein Aagarose beads tumbling at 4° C. for 2 hours.
6. Washing the beads with Triton X-100 lysis buffer×3.
7. Processing the precipitates for blotting with Ni-HRP or with anti-ubiquitin or anti-His antibody as described in self-ubiquitination assay.
8. The substrate ubiquitination will be detected as laddering and/or smearing on the blot.

What is claimed is:

1. A method for detecting ubiquitin ligase (E3) activity in a sample, said method comprising the steps of:
preparing a sample mixture comprising a ubiquitin, a ubiquitin activating enzyme (E1), a ubiquitin conjugating enzyme (E2), a reaction buffer, and the sample;
preparing a control mixture comprising said E1, said E2, a ubiquitin ligase (E3), and said buffer;
incubating said sample mixture and said control mixture under conditions suitable for ubiquitination to occur; and
detecting a presence of ubiquitination product in said sample mixture and in said control mixture after the incubation.

2. The method of claim 1, wherein said E2 is UbCH5b.

3. The method of claim 1, wherein said mixture comprises a panel of E2s.

4. The method of claim 3, wherein said panel of E2s comprises UbCH5b.

5. The method of claim 1, wherein said ubiquitin is untagged ubiquitin.

6. The method of claim 5, wherein the presence of ubiquitination product is detected using an anti-ubiquitin antibody.

7. The method of claim 1, wherein said ubiquitin is tagged ubiquitin.

8. The method of claim 7, wherein said ubiquitin is tagged with a His-tag.

9. The method of claim 8, wherein the presence of ubiquitination product is detected using nickel-HRP.

10. The method of claim 1, wherein said mixture further comprises a ubiquitination substrate.

11. The method of claim 1, wherein said ubiquitin activating enzyme comprises a bacteria extract having E1 activity, wherein said E2 comprises a bacteria extract having UbCH5b activity, wherein said ubiquitin is tagged with a His-tag, and wherein the presence of ubiquitination product is detected using SDS-PAGE followed by blotting and detection with nickel-HRP.

12. The method of claim 1, further comprising:
quantifying the ubiquitination product in said sample mixture and in said control mixture after incubation; and
determining E3 activity in said sample.

13. An assay kit for detecting ubiquitin ligase (E3) activity, said kit comprising:
a ubiquitin activating enzyme (E1);
a ubiquitin conjugating enzyme (E2);
a ubiquitin;
a control ubiquitin ligase (E3);
a reaction buffer; and
an instruction for assay procedure.

14. The kit of claim 13, wherein said E1 is a bacteria lysate having E1 activity, wherein said E2 is a bacteria lysate having UbCH5b activity, wherein said tagged ubiquitin or untagged ubiquitin is a bacteria lysate comprising ubiquitin.

15. The assay kit of claim 14, wherein said ubiquitin comprises a His-tag.

16. The assay kit of claim 15, further comprising nickel-HRP.

17. The assay kit of claim 14, wherein said ubiquitin is one of untagged ubiquitin or tagged ubiquitin.

18. The assay kit of claim 17, further comprising at least one detection agent selected from the group consisting of anti-ubiquitin antibodies, anti-Flag antibodies, anti-myc antibodies, anti-T7 antibodies, anti-HA antibodies, nickel-HRP, and ECL substrates.

19. The assay kit of claim 13, further comprising a sample lysis buffer.

20. The assay kit of claim 13, wherein said reaction buffer comprise Mg⁺⁺ and ATP
